(19) United States
(12) Patent Application Publication
(10) Pub. No.: US 2010/0003206 A1
(43) Pub. Date: Jan. 7, 2010

(54) Cosmetic Composition Containing A Protein And An Enzyme Inhibitor

(76) Inventors: Paolo Ulderico Giacomoni, Commack, NY (US); Abul M. Manirazman, Port Jefferson, NY (US); Peter J. Lentini, Bellmore, NY (US); Gedeon Harvey, New York, NY (US)

Correspondence Address: THE ESTEE LAUDER COS, INC, 155 PINELAWN ROAD, STE 345 S, MELVILLE, NY, 11747, US

(21) Appl. No.: 12/541,439

(22) Filed: Aug. 14, 2009

Related U.S. Application Data
(63) Continuation of application No. 11/030,664, filed on Jan. 6, 2005, now abandoned.
(60) Provisional application No. 60/535,198, filed on Jan. 7, 2004.

(51) Int. Cl. A61K 8/18; A61K 36/00; A61K 36/02; A61K 35/54; A61Q 17/04

(52) U.S. Cl. 424/59; 424/725; 424/195.17; 424/581

(57) ABSTRACT

The present invention is a cosmetic composition comprising a water-soluble protein-based film-forming agent, the film-forming agent having a tertiary structure capable of undergoing a helix-coil transformation, a protease inhibitor in an amount sufficient to protect the protein on the stratum corneum and a cosmetically acceptable vehicle so that the protease inhibitor prolongs the pulling effect of the film-forming agent.

### CROSS-REFERENCE TO RELATED APPLICATIONS

This is a continuation of U.S. patent application Ser. No. 11/030,664 filed Jan. 6, 2005, which claims priority to U.S. Provisional Application No. 60/535,198 filed Jan. 7, 2004.

### FIELD OF THE INVENTION

The present invention relates to a cosmetic composition for application to the skin. In particular, the present invention relates to a cosmetic composition that enhances the appearance of skin by providing a prolonged pulling/tightening effect.

### BACKGROUND OF THE INVENTION

Proteins are an essential part of the human skin composition. In particular, proteins have been identified as playing an important role in the skin aging process as they serve as enzymes, catalyze biological reaction, serve as transport molecules, bind and convey biomolecules to various organs, act as antibodies, act as structural entities and form the major backbone of many tissues. To understand the role of proteins, it is important to understand the composition of the human skin.

The human skin is composed of two main layers. The top layer, the epidermis and its outermost part, the stratum corneum, contains the fibrous protein keratin and functions as a protective cover from the environment. The second layer, the dermis, is connected to the epidermis by way of the basal membrane and links the skin to the rest of the body through the nerves and blood vessels. The dermis is equipped with blood vessels, nerve fibres and lymphatic vessels and comprises a fibrous network of mainly collagen fibres with limited amounts of elastin and reticulin fibres.

The process of cellular differentiation and desquamation occur in the epidermis, a desquamative pluristratified epithelium that consists of several layers. Epidermal differentiation represents a continual and oriented maturation process of cells formed in the bottom layer of the epidermis until they reach the outer layer of the epidermis. Desquamation is the detachment of cells from the outer surface of an epithelium.

Turning to the epidermis, the innermost layer, termed the basal layer, produces epidermal cells. Above the basal layer, a prickle cell layer consists of keratinocytes and several layers of duplicating cells originally produced in the basal layer. A granular layer includes flattened cells containing distinct cytoplasmic inclusions called the keratohyalin granules. In the granular layer, numerous enzymes are released which convert the dead cell material to keratin. Finally, the outer layer, the horny layer, also known as the stratum corneum, contains stacked corneocytes, which are keratinocytes at the final stage of their differentiation, i.e., completely keratinized dead cells. The corneocytes are principally composed of a fibrous matrix containing cytokeratins, surrounded by a very resistant structure, designated the horny or cornified envelope. The stratum corneum is responsible for the barrier function of the epidermis and possesses a selective permeability that, by controlling the exchange of water vapor, oxygen and other gases, ensures a physiological hydration of the skin.

Through the regulation of the number of cells that enter the differentiation process and the number of cells that desquamate in the desquamation process, a constant thickness is maintained to ensure homeostasis of the epidermis. Epidermal differentiation is the result of perfectly coordinated phenomena which will lead to a constant thickness being maintained and which will thus ensure homeostasis of the epidermis.

Bonds formed between the corneocytes and the skin play an important role in the desquamation process. During the normal desquamation process, only the most superficial corneocytes become detached from the surface of the epidermis. From the basal layer to the granular layer, cohesion is provided by the transcellular network defined by the desmosomes and the intermediate filaments of cytokeratins. This network is anchored onto the basal membrane by the hemidesmosomes. In the horny layer, cohesion is provided by the intercellular structures derived from the desmosomes, designated corneosomes or corneodesmosomes, which firmly connect together the horny envelopes of the corneocytes. In the epidermis (non-palmoplantar), the corneodesmosomes are present over the entire corneocyte surface in the lower part of the horny layer, but just the peripheral corneodesmosomes persist in the upper part.

Enzymes produced in the deeper living layers will act sequentially and in a complementary manner, resulting in the final release of the corneocytes at the skin surface. The principal enzymes suspected of taking part in desquamation are glycosidases and proteases. The proteases cannot act alone and a preliminary action involving glycosidases appears to be necessary; however, the proteases are more involved in the desquamation process.

Numerous skin conditions such as xerosis (or dryness of the skin), ichthyoses, psoriasis, certain benign or malignant tumor lesions, and reactive hyperkeratoses have been likened to abnormal desquamation. As one solution, references teach compositions containing proteases to aid in the desquamation and consequently, the exfoliation process. Protease inhibitors are believed to inhibit the breakdown of such proteases that aid the process of desquamation.

Although proteases are believed to aid desquamation, proteases are believed to be responsible for the breakdown of proteins and are able to do so both on the skin and in aqueous solutions. Since proteins provide numerous benefits to the skin, including aiding in the skin aging process, it is important to provide a means of protecting the proteins from proteases. In the stratum corneum, the average protein survives only two days before it is broken down and its chemical parts either are recycled or excreted as waste. See Deciphering The Message of Life's Assembly, David Brown, MD, Washington Post, 1999.

U.S. Pat. No. 6,511,957 issued to Green et al. (hereinafter “patent '957”) teaches compositions comprising a corneocyte envelope protein to provide a protective layer on the skin. The protective layer is provided to mimic the corneocyte envelope in the skin and thereby provide protection against harmful objects from penetrating the skin. Patent '957 further teaches the use of a protease inhibitor to protect the protective layer from degradation by proteases; however, patent '957 limits the proteins to be protected to a class of naturally-occurring water insoluble human proteins as those that will mimic the corneocyte proteins. Patent '957 further teaches the addition of “skin structuring proteins” that are natural proteins considered to be substantive to the skin.

None of the known references recognize the problem that the desquamation process can actually inhibit the effects of proteins before the proteins are able to provide their full beneficial effect. Moreover, none of the references discussed teach the beneficial function of proteins in reducing the appearance of wrinkles. Therefore, there is a need for a cosmetic composition that prolongs the effects of protein-based materials on the skin while providing a novel means of reducing the appearance of wrinkles.

### SUMMARY OF THE INVENTION

The present invention is a cosmetic composition comprising a water-soluble protein-based film-forming agent, the film-forming agent having a tertiary structure capable of spontaneously undergoing a helix-coil transformation. The composition further comprises a protease inhibitor in an amount sufficient to protect the protein on the stratum corneum and a cosmetically acceptable vehicle.

The present invention further comprises a method of transiently reducing the appearance of wrinkles comprising applying a cosmetic or pharmaceutical composition comprising a water-soluble protein-based film-forming agent capable of binding to corneocytes, the film-forming agent forming a film and having a tertiary structure that can spontaneously undergo a helix-coil transformation to cause a pulling effect, a protease inhibitor in an amount sufficient to protect the protein on the stratum corneum, and a cosmetically acceptable vehicle, so that the protease inhibitor prolongs the pulling effect of the film-forming agent.

### DETAILED DESCRIPTION OF THE INVENTION

Bonds tying the corneocytes within the stratum corneum are broken as part of the desquamation process. While not wishing to be bound by any theories, it is believed that film-forming agents bind to the corneocytes. Therefore, it is believed that as the bonds are broken during desquamation, these film-forming agents are also destroyed.

Since such bond destruction is linked to the beneficial process of desquamation in the skin, it is anti-intuitive and unobvious to attempt to prevent destruction of the bonds by protecting against desquamation. In the present invention, it was surprisingly found that protease inhibitors may be used in the stratum corneum to protect against destruction of the bonds tying the corneocytes within the stratum corneum and therefore protect a certain class of film-forming agents applied onto the stratum corneum. Through such protection, the cosmetic benefits of such film-forming agents are also prolonged.

The present invention comprises a cosmetic composition comprising a water-soluble protein-based film-forming agent having a tertiary structure capable of spontaneously undergoing a helix-coil transformation (hereinafter “film-forming agent”). The term “tertiary structure” means the three dimensional arrangement of all atoms in the protein. Specifically, the tertiary structure refers to the shape that results from the process of folding the protein backbone chain along with secondary structures already formed. The foldings are superimposed on the secondary structures during the synthesis of the protein. For this reason, a tertiary structure is often referred to simply as a fold.

The term “spontaneously undergo a helix coil transformation” refers to the automatic or spontaneous transformation from a linear state to a tertiary state or vice versa, upon contact with the skin and without the aid of denaturing agents. Specifically, although most proteins undergo transformation from a primary linear state to a tertiary state, many proteins are either not capable of unfolding or require denaturing agents to unfold. See Simple Models for Protein Structure and Folding, Nicholas Donald Socci, 1992. It has been discovered that certain classes of proteins are similar to proteins in their nascent state so that the proteins spontaneously undergo helix-coil transformation to fold and unfold, thereby causing a pulling effect on the skin, due to a change in environment, such as when a cosmetic vehicle containing the protein changes such as by drying or evaporating. Such classes of proteins are preferably intended to include proteins derived from plant, vegetable, algae or bacterial sources, and biotechnologically synthesized proteins, although animal sources that provide film-forming agents may also be used. Such proteins include but are not limited to, Protein derived from silk (Silk Crystal from Charles B. Chrystal Co. Inc.), Almond Protein (Polylift from Silab), Protein derived from milk (Lactofirm from Barnet Products), Protein derived from Whey (Versapro E from Davisco), Soy Protein, Tobacco Leaf Protein, Protein derived from bacteria, Fibrous protein from beans, and Protein derived from egg whites such as albumins. Particularly preferred are the Proteins derived from silk such as Silk Crystal.

A “pulling effect” as used herein refers to, among other effects, a tightening of the skin. The tightening is measured for example by a Gas Bearing Electrodynamometer (GBE) as described in Example 1. The protein based film-forming agent exhibits a pulling effect as defined in the present invention if skin tightening is increased by a tightening factor TF of at least about 2% to about 100%, preferably from about 3% to about 60% and most preferably from about 4% to about 40%.

It is believed that film-forming agents that can spontaneously undergo a helix-coil transformation, cause a pulling effect on the skin and thereby maintain the tautness of the skin. In particular, as the film-forming agent transforms from one state to another in the stratum corneum, the transformation creates a pulling effect that keeps the skin taut. While not wanting to be bound to any theories, it is believed that the film-forming agent binds to corneocytes while causing the pulling effect. The pulling effect therefore minimizes the appearance of lines and wrinkles. The present invention provides a surprising new combination that prolongs the pulling effect on the skin to prolong the minimization of lines and wrinkles.

The film-forming agent is provided in an amount sufficient to cause a pulling effect on the skin. The composition is formulated to be suitable for topical application to mammalian skin, hair and/or nails. The amount will vary depending on the formulation and the performance desired. In general, an amount of from about 0.0001% to about 90% by weight of the agent is used, preferably from about 0.001% to about 80%, more preferably from about 0.01% to about 50%, and most preferably from about 0.01% to about 5%.

In a preferred embodiment, the composition further comprises a protease inhibitor in an amount sufficient to protect the film-forming agent on the stratum corneum. The protease inhibitors have surprisingly been found to prevent desquamation in the stratum corneum. As a result, the protease inhibitors protect the protein-based film-forming agent from proteases that would degrade the film-forming agent on the stratum corneum, thereby prolonging the pulling effect of the film-forming agent.

Preferred protease inhibitors include but are not limited to extracts from plants, animals, oils, as well as synthetic and natural molecules. For example, protease inhibitors include A E Complex, a blend of passion flower, black current and grape leaf extracts (Barnet Products), A.L.E., Anti-Leuco Elastase which is Dextran and tetrapeptide-1 (Vincience), hops extract (Alpha Lupulin from Indena), hydrolized Ulva Lactuca Extract (Aosaine from Secma), areca catechu (Betel Nut) extract (Giepl), Blue Algae Extract (Collaborative Labs), Egg extract (Orew Plus from Inovatech Inc.), white birch extract (Premier Specialties Inc.), allicin, aprotinin, leupeptin, bestatin, pepstatin A, N-alpha-p-tosyl-L-lysyl-chloromethyl-ketone and other amino acid analogs and trypsin-chimotrypsin inhibitors.

The protease inhibitor is provided in an amount sufficient to protect the film-forming agent. In general, an amount of from about 0.01 μM to about 100 mM and most preferably from about 0.1 μM to 1 μM is used in the total formulation of the present invention. If used in the crude form, the film-forming agent is used in an amount of from about 0.001% to about 90% by weight. Preferably, about 0.001% to about 60% and most preferably, an amount of from 0.01% to about 5% is used.

An important aspect of the present invention is the need for the film-forming agent to remain on the stratum corneum to provide the beneficial pulling effects in the skin in order to reduce the appearance of wrinkles. If the film-forming agent were allowed to penetrate the skin more deeply, the beneficial proteins would be destroyed rapidly. Further, the anti-penetrant promotes a more uniform distribution of the film-forming agent on the stratum corneum. Therefore, in an alternative embodiment, the present composition comprises an anti-penetrant to keep the film-forming agents on the stratum corneum. Examples of anti-penetrants include large polymers with molecular weights too high to penetrate deeply into skin. By virtue of providing preferential solubility, an ingredient may be more soluble in a given polymer than in skin tissue components. Therefore, if the material shows better solubility in the polymer system than in skin tissue components, and if the polymer is too large to penetrate, then the ingredient in question will not penetrate either.

Any number of polymeric materials are contemplated as being useful as the anti-penetrant of the present invention based upon the criteria discussed in the previous paragraph. General categories of materials exhibiting these associative properties and exhibiting this attenuation include but not limited to, linear and branched polyurethanes, polyethylenes, polyacrylates, polyquaternary cellulosics, and various block and comb and elastomeric copolymers of silicone, pyrrolidone, acrylic acid, methylacrylic acid, and the like. Examples of naturally-occurring materials include carageenans, alginates, pectins, gums, resins, roesins, zein, mannans and starches. In the preferred embodiment of the invention, the anti-penetrant comprises a polyurethane polyol prepolymer commercially available from Barnet, Inc.

The anti-penetrant may be present in an amount sufficient to prevent or inhibit the film-forming agent from penetrating beyond the uppermost layers of the skin, e.g., the stratum corneum. In particular, the anti-penetrant is present in an amount of about 0.2 to about 20.0 percent by weight. Preferably, the compositions of the invention contain about 5.0 to about 15.0 percent by weight anti-penetrant. Most preferably, the compositions contain about 10 percent by weight of the anti-penetrant.

The present composition also optionally includes anti-exfoliants to aid in preventing desquamation to protect the effects of the film-forming agent. Examples of anti-exfoliants include tulip extracts such as extract of narcissus bulb (IBR-Dormin™ from Nu Skin International, Inc.) and cholesterol.

The composition further comprises a cosmetically acceptable vehicle that is suitable for topical application to skin, hair and/or nails. For this purpose, the composition may comprise ingredients adapted to form a cream, gel, emulsion, liquid, suspension, nail coating, skin oil or lotion. Ingredients which may be included in the composition include, for example, moisturizing agents, astringent agents, non-protein film-forming materials, surfactants, emollients, humectants, moisturizers, sunscreens, pigments or other proteins and/or fibers. For example, emollients which may be used in the composition include, but are not limited to, stearyl alcohol, glyceryl monoricinoleate, glyceryl monostearate, propane-1,2-diol, butane-1,3-diol, mink oil, cetyl alcohol, isopropyl isostearate, stearic acid, isobutyl palmitate, isocetyl stearate, oleyl alcohol, isopropyl laurate, hexyl laurate, decyl oleate, octadecan-2-ol, isocetyl alcohol, cetyl palmitate, dimethylpolysiloxane, di-n-butyl sebacate, isopropyl myristate, isopropyl palmitate, isopropyl stearate, butyl stearate, polythylene glycol, triethylene glycol, lanolin, sesame oil, coconut oil, arrachis oil, castor oil, acetylated lanolin, fatty alcohols, petroleum, mineral oil, butyl myristate, isostearic acid, palmitic acid, isopropyl linoleate, lauryl lactate, myristyl lactate, decyl oleate, myristyl myristate.

Humectants which may be used include, but are not limited to, polyhydric alcohols including glycerol, polyalkylene glycols and alkylene polyols and mixtures thereof, hyaluronic acid, urea, glycerin, sorbitol, sodium 2-pyrrolidone-5-carboxylate, soluble collagen, dibutylphthalate and gelatin.

For purposes of the present invention, vehicles are substances that can act as diluents, dispersants, or solvents for the proteins or polypeptides. The vehicle is preferably one which can aid formation of a protective layer on the skin. The vehicle or vehicles can comprise from about 1 to about 99.9%, preferably from about 50 to about 99.5%, more preferably from about 70 to about 99.3% and most preferably from 80 to 90% by weight of the compositions.

The composition of the present invention may also contain additional cosmetic ingredients that add to the aesthetics of performance of the present invention. For example, pigments, thickeners, powders and fragrances may be used to increase the aesthetic appeal of the present invention.

Pigments may include but are not limited to titanium dioxide, micas, iron oxides, barium lake, calcium lake, aluminum lake, bismuth oxychloride, zirconium lake and calcium oxides.

Thickeners may include starch, gums such as gum arabic, kaolin or other clays, hydrated aluminum silicate, fumed silica, carboxyvinyl polymer, sodium carboxymethyl cellulose or other cellulose derivatives, ethylene glycol monostearate and sodium alginates.

Powders may include chalk, talc, fullers earth, colloidal silicon dioxide, sodium polyacrylate, tetra alkyl and/or trialkyl ammonium smectites and chemically modified magnesium aluminum silicate.

The present composition may optionally comprise a fragrance in an amount sufficient to make the composition more appealing to the consumer. Preferably, the perfume is in the amount of from about 0.01 to about 10% by weight of the composition.

In addition to the elements described above, the skin care compositions of the present invention may include skin care actives. Actives are defined as skin or hair benefit agents other than emollients and other than ingredients that merely improve the physical characteristics of the composition. Examples of actives that may be useful include, but are not limited to, agents for the eradication of age spots, keratoses and wrinkles, analgesics, anesthetics, anti-acne agents, antibacterials, antiyeast agents, antifungal agents, antiviral agents, antidandruff agents, antidermatitis agents, antipruritic agents, antiemetics, antimotion sickness agents, anti-inflammatory agents, antihyperkeratolytic agents, anti-dry skin agents, antiperspirants, antipsoriatic agents, antiseborrheic agents, hair conditioners and hair treatment agents, antiaging agents, antiwrinkle agents, antiasthmatic agents and bronchodilators, sunscreen agents, antihistamine agents, skin lightening agents, depigmenting agents, vitamins, corticosteroids, self-tanning agents, hormones, retinoids such as retinoic acid and retinol, topical cardiovascular agents, clotrimazole, ketoconazole, miconozole, griseofulvin, hydroxyzine, diphenhydramine, pramoxine, lidocaine, procaine, mepivacaine, monobenzone, erythromycin, tetracycline, clindamycin, meclocyline, hydroquinone, minocycline, naproxen, ibuprofen, theophylline, cromolyn, albuterol, topical steroids such as hydrocortisone, hydrocortisone 21-acetate, hydrocortisone 17-valerate, and hydrocortisone 17-butyrate, betamethasone valerate, betamethasone diproprionate, triamcinolone acetonide, fluocinonide, clobetasol, proprionate, benzoyl peroxide, crotamiton, propranolol, promethazine, vitamin A palmitate, vitamin E acetate and mixtures thereof. Incorporation of skin care actives into such a composition may benefit the activity of the active by prolonging its presence on the skin, thereby enhancing its efficacy.

### Methods for Regulating Skin Aging and Other Disorders

The present inventive compositions are particularly useful as anti-aging products. Specifically, as the film-forming agent transforms from one state to another on the epidermis, the conformational change of the film-forming agent creates a pulling effect that keeps the skin taut and reduces the appearance of lines and wrinkles. The novel compositions and methods of the present invention protect the film-forming agent while preventing the penetration of the film-forming agent itself into the inner layers of the skin, thereby prolonging the pulling effect on the skin.

The present invention also relates to methods of improving the appearance of skin and methods of prolonging a pulling effect. The appearance of skin is improved by, for example, regulating and reducing the appearance of skin aging. As used herein, “skin aging” includes skin atrophy and means the thinning and/or general degradation of the dermis caused by free radical damage which is often characterized by an alteration and degeneration of collagen and/or elastin. Skin aging may be caused by either intrinsic or extrinsic factors such as natural chrono-aging, photo damage, burns, or chemical damage. As used herein, “regulating skin aging” means preventing, retarding, arresting, treating, or reversing the process of skin aging in mammalian skin. Examples of regulating skin aging include but is not limited to reduction of the appearance of lines and wrinkles, reduction of the effect of skin atrophy and reduction of the appearance of thinning.

The present invention further comprises methods of regulating other skin disorders with the addition of an active (including those that are peptidic or polypeptidic in nature), including but not limited to skin disorders such as dry skin, severe dry skin, xerosis, dandruff, keratoses, psoriasis, eczema, age spots, lentigines, melasmas, blemished skin, hyperpigmented skin, hyperkeratotic skin, inflammatory dermatoses and age-related skin changes. Such a method comprises administering or topically applying to the skin a safe and effective amount of the combination of the present invention, which further comprises the active component. The amounts of the components in the compositions will vary widely depending upon the level of skin aging already in existence in the subject (if such exists), the rate of further aging, and the level of regulation desired.

The regimen of application will depend on the ultimate intended use of the composition. For example, the composition may be applied on an as needed basis, to disguise a wrinkle, line or other symptom of skin aging. Alternatively, a chronic application may be desired, particularly with a composition containing an active agent to regulate skin aging and to treat other skin maladies described above. A preferred amount of treating the skin is via topical application of a safe and effective amount of the novel composition to regulate skin aging and to treat other skin maladies described above.

For chronic topical application, the amount of the composition and the frequency of topical application to the skin can vary widely, depending upon the particular skin disorder and the severity thereof. It is suggested as an example that topical application range from about once per week to about 4 or 5 times daily, preferably from about 3 times a week to about 3 times daily, most preferably about once or twice per day. The compositions will comprise from about 0.01 to 90%, preferably from about 1 to 20%, and most preferably from about 1 to 5% of the active components. However, it should be noted that it is well within the purview of the skilled artisan, such as a dermatologist or other health care provider, to regulate pharmaceutical dosages according to patient needs.

The following examples further illustrate the novel compositions and methods of the present invention, but the invention is not limited thereto.

### Example 1

The tightening of the skin is assessed by a Gas Bearing Electrodynamometer (GBE). The GBE measures the force acting on the skin and the displacement of the skin due to that force. The displacement represents the strain of the hysteresis loop produced by the movement of the GBE probe, and the force applied is the stress of the hysteresis loop. The average slope of the hysteresis loop is proportional to 1/DSR (Dynamic Spring Rate) which is a measure of skin softness. Skin tightening “t” is assessed by comparing the displacement before (DB) and after (DA) product treatment and is expressed as t=(DB−DA)/DB. The residual tightening RT is expressed as RT=[t(3 hours)]/[t(0 hours)].

The formula tested to assess the tightening of the skin by film-forming agents and the effect of a protease inhibitor, an Egg extract (Orew Plus from Inovatech Inc.), is provided below in Table 1 and the results are reported in Tables 2 and 3. Specifically, the combination of the protease inhibitor with the film-forming agent provides the desired skin pulling/tightening. The formula in Table 1 below provides a unique combination that protects the film-forming agent to prolong the skin pulling effect to keep the skin taught for a longer duration, as is shown in the results in Table 2. The result is a reduction in the appearance of symptoms of aging for a longer period of time.

TABLE 1
Cream

| | |
|---|---|
| Glycerine | 1% |
| Magnesium Ascorbyl Phosphate (Ascorbyl PM) | 1% |
| Water | Q/s |
| Methyl Gluceth 20 (Glucam E-20) | 2% |
| Caffeine | 0.2% |
| Caprylyl Glycol (Lexgard O) | 0.8% |
| Ethylenediamine Disodiumacetate (EDTA-Na2) | 0.1% |
| Sucrose | 1% |
| Butylene Glycol | 1% |
| Dimethicone Copolyol (Biowax ™ 754) | 5% |
| Water | 5% |
| Beta-Hydroxide | 1.5% |
| Butylene Glycol | 1% |
| Xanthan Gum (Ketrol 1000) | 0.3% |
| Linoleic Acid (Emersol 315) | 0.1% |
| Isoprene Glycol | 2% |
| Butylated hydroxyanisole (BHT) | 0.1% |
| PEG-60 hydrogenated castor oil (Emalex-HC 60) | 2% |
| Vitamin E | 1% |
| Phenoxyethanol (Emeressence 1160) | 0.5% |
| Green Tea | 2% |
| Saxifraga Sarmentosa Extract/Grape | 1% |
| Extract/Butylene Glycol/water (PHYTOCLAR ™ II) | |
| NaOH solution | Q/s |
| Protein derived from milk, Lactofirm | 2% |
| Whey Protein (MPC II) | 0.2% |
| Pyrus Mallas (apple) Extract (Micromel) | 1% |
| Vitamin A Palmitate | 0.1% |
| Wheat Flour Lipids (Cennimides cerp 50) | 0.1% |
| Soybean (Glycerine Soja) Sterol (General 122) | 0.1% |
| Egg Extract (Orew Plus) | 0.5% |

TABLE 2

| | Tightening Time | | | | |
|---|---|---|---|---|---|
| Formula | 0 | 1 hour | 2 hours | 3 hours | 4 hours |
| 0.5% | 17% | 17% | 13% | 9% | 7% |
| Protease | | | | | |
| Inhibitor | | | | | |
| Control | 14% | 11% | 11% | 5% | 0% |
| (without | | | | | |
| the | | | | | |
| presence | | | | | |
| of | | | | | |
| Protease | | | | | |
| Inhibitor) | | | | | |

As can be seen in Table 2 above, the presence of a protease inhibitor, Egg Extract (Orew Plus) with a Protein derived from Milk (Lactofirm), a water-soluble protein-based film-forming agent having a tertiary structure capable of spontaneously undergoing a helix-coil transformation, prolongs the tightening effect of the film-forming agent. Specifically, at 4 hours after application of the formula, skin tightening is increased by a tightening factor TF of 7%.

Table 3 below further shows the effect of a protease inhibitor, reporting the residual tightening versus time.

TABLE 3

| | Residual Tightening At Time | | | | |
|---|---|---|---|---|---|
| Formula | 0 | 1 hour | 2 hours | 3 hours | 4 hours |
| 0.5% | 100 | 100 | 76 | 53 | 41 |
| Protease | | | | | |
| Inhibitor | | | | | |
| Control | 100 | 78 | 78 | 35 | 0 |
| (without | | | | | |
| the | | | | | |
| presence | | | | | |
| of | | | | | |
| Protease | | | | | |
| Inhibitor) | | | | | |

As seen in Table 3, the residual tightening at 4 hours remains at 41, as opposed to 0 hours without the presence of a protease inhibitor.

### Example 2

The compositions of the present invention may also be formulated as a gel composition. Table 4 provides an example of such a composition.

TABLE 4
Gel

| Deionized Water | Q/s |
|---|---|
| Almond Seed Extract (Polylift) | 20% |
| Glycerine | 2% |
| Methylchloroisothiazolinone (Kathon ™ CG) | 0.1% |
| Ethylenediamine Disodiumacetate (EDTA-Na2) | 0.1% |
| White Birch Extract | 0.5% |
| Polyolprepolymer 2 | 10% |
| Tulip extract (IBR Dormin) | 1% |

It should be understood that the specific forms of the invention herein illustrated and described are intended to be representative only. Changes, including but not limited to those suggested in this specification, may be made in the illustrated embodiments without departing from the clear teachings of the disclosure. Accordingly, reference should be made to the following appended claims in determining the full scope of the invention.

What is claimed is:

1. A method of providing a prolonged a pulling effect on the surface of the skin, comprising applying to the skin a cosmetic composition comprising a water-soluble protein-based film-forming agent in combination with a protease inhibitor, wherein the film-forming agent is capable of binding to corneocytes and has a tertiary structure that can spontaneously undergo a helix-coil transformation to cause a pulling effect on the skin, and wherein the protease inhibitor is present in an amount sufficient to protect against destruction of bonds formed between the protein-based film-forming agent and the corneocytes within the stratum corneum, thereby prolonging the pulling effect of the film-forming agent on the skin.

2. The method of claim 1, wherein the protease inhibitor is present at an amount ranging from about 0.01% to about 5%.

3. The method of claim 1, wherein the cosmetic composition tightens the skin by a skin tightening factor of about 7% after 4 hours of application onto the skin.

4. The method of claim 1 wherein the protein-based film-forming agent is selected from the group consisting of plant, vegetable, algae, bacterial, animal-derived, or biotechnologically synthesized proteins.

5. The method of claim 1 wherein the protein-based film-forming agent is selected from the group consisting of silk protein, almond protein, milk protein, whey protein, soy protein, tobacco leaf protein, bacterial protein, egg white protein and fibrous protein from beans.

6. The method of claim 1 wherein the protease inhibitor is selected from the group consisting of hops extract, passion flower, black current and grape leaf extract, hydrolized ulva lactuca extract, betel nut extract, blue algae extract, egg extract, white birch extract, allicin, aprotinin, leupeptin, bestatin, pepstatin A, N-alpha-p-tosyl-L-lysyl-chloromethyl-ke-tone and trypsin-chimotrypsin inhibitors.

7. The method of claim 1, wherein the cosmetic composition further comprises an anti-penetrant for keeping the protein-based film-forming agent on the stratum corneum and preventing it from penetrate the skin.

8. The method of claim 7, wherein the anti-penetrant is a polymer with a sufficiently high molecular weight that it cannot penetrate into the skin, and wherein said polymer is selected from the group consisting of linear and branched polyurethanes, polyethylenes, polyacrylates, polyquaternary cellulosics, block and comb and elastomeric copolymers of silicone, pyrrolidone, acrylic acid, methylacrylic acid, carageenans, alginates, pectins, gums, resins, roesins, zein, mannans, starches, and combinations thereof.

9. The method of claim 7, wherein the anti-penetrant is a polyurethane polyol prepolymer.

10. The method of claim 1 wherein the cosmetic composition further comprises an anti-exfoliant for preventing desquamation and protecting the pulling effect of the protein-based film-forming agent on the skin.

11. The method of claim 1 wherein the cosmetic composition further comprises a skin care active.

12. The method of claim 11 wherein the skin care active is selected from the group consisting of anti-aging, anti-acne and sunscreen agents.
